(19) United States
(12) Patent Application Publication
(10) Pub. No.: US 2021/0319910 A1
(43) Pub. Date: Oct. 14, 2021

(54) CONTACT TRACING OF EPIDEMIC-INFECTED AND IDENTIFICATION OF ASYMPTOMATIC CARRIERS

(71) Applicant: Dualiti Interactive LLC, Newton, MA (US)

(72) Inventors: Abraham Seidmann, Newton, MA (US); Yaniv Ravid, New York, NY (US)

(21) Appl. No.: 17/221,329

(22) Filed: Apr. 2, 2021

Related U.S. Application Data
(60) Provisional application No. 63/008,287, filed on Apr. 10, 2020.

(51) Int. Cl. G16H 50/80; G16H 10/60; G16H 50/30; G06Q 50/26; G16H 15/00; G16H 50/20; H04W 4/029; G08B 5/22; G01S 19/42

(52) U.S. Cl. CPC G16H 50/80; G16H 10/60; G16H 50/30; G06Q 50/26; G16H 40/20; G16H 50/20; H04W 4/029; G08B 5/22; G01S 19/42; G16H 15/00

(57) ABSTRACT

A method for contact tracing of a carrier of infection within a population identifies the carrier of the infection and acquires recorded tracking information that shows geographical location and timing for each of a plurality of members of the population within a predetermined time period. From the acquired tracking information, at least a first geographic location visited by the carrier and a corresponding first time interval for the carrier visit are identified and at least a second individual determined to have been present at the first visited geographical location for a second time interval that at least partially overlaps the first time interval is identified. A corresponding infection risk value for the second individual is calculated. A first signal having message content that alerts the second individual to the calculated infection risk value is transmitted. The transmitted message content displays on a personal communications device associated with the second individual.

### CROSS REFERENCE TO RELATED APPLICATIONS

Reference is made to, and priority is claimed from, commonly assigned U.S. Ser. No. 63/008,287, filed as a provisional patent application on 10 Apr. 2020, entitled “CONTACT TRACING OF EPIDEMIC-INFECTED AND IDENTIFICATION OF ASYMPTOMATIC CARRIERS” in the names of Abraham Seidmann and Yaniv Ravid, incorporated herein in its entirety.

### FIELD

The present disclosure relates to a computerized system and methods for tracing interactions having a high likelihood of transmitting infection within a population and more particularly for identifying asymptomatic carriers in a population.

### BACKGROUND

In the public health field, contact tracing is the process used for identifying persons who may have come into contact with an infected person (“contacts”) and can include subsequent collection of further information about these contacts. By systematically tracing the contacts of infected individuals, testing these contacts for infection, treating the infected and tracing their contacts in turn, public health aims to reduce infections in the population. Among diseases for which contact tracing is commonly performed are tuberculosis, vaccine-preventable infections like measles, sexually transmitted infections (including HIV), blood-borne infections, some serious bacterial infections, and so-called novel infections (e.g. SARS-CoV and SARS-CoV-2, or COVID-19). Goals of the tracing effort include interrupting ongoing transmission and spread of infection, alerting contacts to possibility of infection and opportunities for preventive care, offering diagnosis and treatment to those infected, helping to prevent re-infection, and collecting data related to the epidemiology of a disease in a given population.

A goal of effective epidemic management and control within a population is to try and rapidly identify those who are infected and suffering from disease symptoms as well as anyone who may have been in earlier contact with infected individuals and, therefore, have potentially been exposed to a disease organism. There is also a pressing need to identify, as early as possible, those individuals within the community who may not exhibit symptoms but who may be acting as carriers of the infection (“Asymptomatic Carriers”). Contact tracing for individuals exposed to infection can help to identify and isolate the disease for treatment and help to protect others within the population.

Current contact tracing processes for infected persons are manual, slow, inaccurate, and do not readily scale. As recent history shows, infection can spread exponentially, far outpacing manual methods for tracing and isolating carriers of the infection. In some ways, technological advancement has compounded the problem, making it easier for infection to spread in a more highly mobile society that facilitates interaction over larger populations. Contact tracing techniques have not kept pace with the relative speed with which infection can spread, presenting considerable concern to those who work with public health.

The contact tracing task is particularly difficult with asymptomatic carriers, who may unwittingly come in contact with numerous others and unwittingly spread the disease. Statistical estimates have shown that around a quarter to half of the carriers of the recent corona virus, for example, are asymptomatic, which greatly complicates the task of identifying (and isolating) any individual acting as a potential carrier.

In a similar aspect, other types of infection that can be a concern include the spread of computer viruses and malware through nodes on a network. In the fields of data analysis and information management, tracing network interactions related to the spread of malignant software may benefit from techniques used for health-related tracing and identification.

Thus, it can be appreciated that there would be benefits to contact tracing methods that provide intelligent proactive infection tracing, useful in a number of fields.

### SUMMARY

The Applicants address the problem of contact tracing for infected individuals and those acting as carriers of disease in a population.

Particular embodiments of the present disclosure provides a system and method for contact tracing under epidemic conditions and for notifying individuals of likely exposure to a disease condition or likely status as a carrier of an infective organism. Solutions described herein can help to identify those in need of isolation, potential testing and treatment and can help to inform and educate individuals who may be at a higher risk due to exposure.

The present disclosure describes an improved form of contact tracing, which reacts to growing infection rates within a population and proactively determines when an individual among a population is a likely asymptomatic carrier. Such individuals can be suitably informed of likely carrier status and steps taken to help restrict or slow the spread of infection accordingly.

Embodiments of the present disclosure can include a data management system which stores information about the population such as individuals' physical location, past medical history and other characteristics. Information is added to update the system on a regular basis. Maintenance of the information may be done manually by individuals, automatically through a separate digital procedure, by system administrators with information management privileges, or through any other suitable digital or physical process.

A method of the present disclosure also consists of a regularly executed process which analyzes the stored information. This could be executed on a single process or a group of processors, which takes known information for each of a set of individuals and processes it to identify communities with greater infection spread as well as determine the likelihood that a member of the population is an asymptomatic carrier. Records indicating new infections may be used to flag certain communities that appear to show a trend toward critical infection rates, and among which groups of individuals future exposures are most likely to proliferate. Using a quantifiable definition for apparent interactions between individuals, given the available information, the process classifies which interactions have likely caused individuals to become asymptomatic, who may present a risk to their community.

Additionally, the present disclosure comprises a digital process which acts on this information in order to proactively mitigate future spread of infections. The process could be run on a single or a group of processors and immediately alert individuals of their risk to be asymptomatic, distribute the list of all asymptomatic individuals to public health officials prior to alerting individuals, or take any other form of action on the basis of the analysis results.

Following such alerts, the system of the present disclosure can use new information regarding its earlier conclusions to improve itself. For example, after a notification of being asymptomatic, members of the population may verify this information by getting tested. The results of such action to confirm the system's findings could be used to improve its future rates of accuracy, specificity, or any other practical measure of successfully achieving its objective.

An embodiment of the present disclosure can include a computerized system that performs the needed tasks and processes described. The system gathers the necessary information to perform these tasks, executes the procedures at the correct order and time intervals, presents the results and visibility into programs to privileged users, individuals in the population, health officials, and to any other potential users via a variety of interfaces and visual channels.

According to an embodiment of the present disclosure there is provided a method for contact tracing of a carrier of a communicable infection within a population, the method comprising:
a) identifying the carrier of the infection according to received medical data;
b) acquiring recorded tracking information that shows geographical location and timing for each of a plurality of members of the population within a predetermined time period;
c) identifying, from the acquired tracking information at least a first geographic location visited by the carrier and a corresponding first time interval for the carrier visit;
d) identifying, from the acquired tracking information, at least a second individual determined to have been present at the first visited geographical location for a second time interval that at least partially overlaps the first time interval;
e) calculating a corresponding infection risk value for the second individual;
f) transmitting a first signal having message content that alerts the second individual to the calculated infection risk value; and
g) displaying the transmitted message content on a personal communications device associated with the second individual.

### DRAWINGS

The foregoing and other objects, features, and advantages of the invention will be apparent from the following more particular description of the embodiments of the disclosure, as illustrated in the accompanying drawings.

FIG. 1 is a schematic diagram showing an example of carrier tracing interactions on Day 1.

FIG. 2 is a schematic diagram showing an example of carrier tracing interactions on Day 2.

FIG. 3 is a schematic diagram showing an example of carrier tracing interactions on Day 3.

FIG. 4 is a logic flow diagram that relates the response of an intelligent proactive infection tracing process to conventional practice.

FIG. 5 is a flowchart displaying the steps and processes that improve the operational and medical alertness of the public health system.

FIG. 6 is a flow diagram that explains the abstract processes presented in FIG. 5 with greater detail.

FIGS. 7A and 7B are flowcharts describing the continuous utility of the system as time progresses.

FIG. 7C is a logic flow diagram showing a process for asymptomatic carrier identification and notification according to an embodiment of the present disclosure.

FIGS. 8A-8C are examples of messages generated and transmitted as potential notification displays for an individual determined to be at risk, according to an embodiment of the present disclosure.

FIGS. 9A and 9B show an exemplary command and control configuration display that displays the information gathered, processed, and analyzed by the system.

FIG. 10 is an interface display that shows a summary of risk analysis results for the population.

FIG. 11 is an interface display that shows exemplary statistics and recommendations for value adjustment.

FIGS. 12A and 12B show exemplary ROC charts for accuracy in detecting true positives.

### DETAILED DESCRIPTION

The following is a detailed description of exemplary embodiments, reference being made to the drawings in which the same reference numerals identify the same elements of structure in each of the several figures.

Where they are used in the context of the present disclosure, the terms “first”, “second”, and so on, do not necessarily denote any ordinal, sequential, or priority relation, but are simply used to more clearly distinguish one step, element, or set of elements from another, unless specified otherwise.

The term “set”, as used herein, refers to a non-empty set, as the concept of a collection of elements or members of a set is widely understood in elementary mathematics. The term “subset”, unless otherwise explicitly stated, is used herein to refer to a non-empty proper subset, that is, to a subset of the larger set, having one or more members. For a set S, a subset may comprise the complete set S. A “proper subset” of set S, however, is strictly contained in set S and excludes at least one member of set S.

The term “population” has its conventional meaning, relating to a group of individuals. The term “virus” is used for illustration in particular examples given herein; in the context of the present disclosure, terms such as “virus”, “infectious agent”, “germ”, or “infection” can be interchangeably used to indicate the communicable agent that is transmitted between individual members of the population and appears to be responsible for the ensuing illness or significant impairment of normal health or function in the population.

The phrase “in signal communication” as used in the application means that two or more devices and/or components are capable of communicating with each other via signals that travel over some type of signal path. Signal communication may be wired or wireless. The signals may be communication, power, data, or energy signals which may communicate information, power, and/or energy from a first device and/or component to a second device and/or component along a signal path between the first device and/or component and second device and/or component. Signal paths may include physical, electrical, magnetic, electromagnetic, optical, wired, and/or wireless connections between the first device and/or component and second device and/or component. Signal paths may also include additional devices and/or components between the first device and/or component and second device and/or component.

In the context of the present disclosure, the terms “tracing” and “tracking” can be used equivalently. Contact tracing, as described in the background, is a form of tracking that relates to patterns of contact that can spread an infection.

For execution and orchestration of the various tasks and capabilities listed hereinabove, embodiments of the present disclosure can employ trained logic or learned logic, equivalently termed “machine learning” an aspect of Artificial Intelligence (AI) by which control logic is trained by monitoring its own performance and making adjustments over time, and can utilize various tools and capabilities. Trained or machine-learned logic can be distinguished from conventional programmed logic that is formulated based on a formal instruction language that is used to specify particular data operations performed by a logic processor. In various embodiments, the processing logic can include portions of executable code that have been generated using conventional procedural programming logic that provides a predictable response according to received inputs, as well as other portions of executable code that have been generated using machine learning techniques that are characterized as model-based and probabilistic, based on training using multiple examples, and provide solutions derived from heuristic processes. Typically, machine learning techniques employ multi-layer neural network architectures and can employ associated techniques that detect higher-level patterns from volumes of low-level data such as “deep learning”, for example. Thus, in the context of the present disclosure, terms such as “machine learning”, “artificial intelligence”, “deep learning”, and “neural networks” can be used equivalently to describe trained logic from various related aspects.

The learned logic, which can be considered the machine learning algorithm output, can be implemented using a trained neural network or using a statistical model-based method that utilizes data that has been collected from any of a number of sources.

In public health and safety, epidemics have brought severe strains onto healthcare providers as well as members of the public. Healthcare systems around the world have recently found themselves nearly overcome, leading to possibly further loss of life as well as other hardships, such as mental and physical damage to both patients and health providers.

As considered in the present disclosure, the spread of infectious conditions can relate not only to viral or microbial epidemics, but also to the spreading of malicious software among a network of computers, for example. For the sake of description, the present disclosure focuses on epidemic or pandemic situations; however, the applicability of methods of the present disclosure to other fields can be readily seen. For example, the approach described herein can be used to identify contact of an infectious agent, such as a microbe, virus, or other self-replicating entity including a segment of malicious computer code, conveyed by a carrier entity, which could be a person, animal, or data message, and interacting with other members of a population in different ways, at different times and locations.

Methods of the present disclosure target shortcomings of the conventional methods of mitigation for infection spread which have been so far developed and used to attack and restrain viral propagation among communities. One particular method, contact tracing, has proven to be limited and narrow in scope when executed using conventional approaches. Methods of the present disclosure address the problem to assist healthcare systems and providers by focusing on at least one aspect of viral spread which has been, thus far, largely ignored in the medical technology field. Solutions described herein can help to reduce the strain on healthcare systems and on society at large by identifying asymptomatic carriers amid the population.

It has been acknowledged that transmission of communicable infection by those who do not appear to have symptoms, that is, asymptomatic carriers, presents particular challenges not met using conventional contact tracing approaches. In the recent COVID-19 pandemic, it has been held that a significant amount of transmission was conveyed by individuals either without symptoms or who may have had only mild symptoms. Contagious carriers who do not exhibit observable symptoms can make it difficult to carry out effective programs of hygiene, screening, and reduced contact or isolation.

Embodiments of the present disclosure can provide data management, digital processes, methods and computer systems which are helpful to achieve this objective. Other embodiments of the present disclosure include interfaces and notification systems to present and distribute these methods and processes to necessary parties, such as municipal command and control groups that oversee epidemic control and related societal events.

Among a population where viral spread is ongoing, certain communities may undergo greater rates of infection that others. This infection occurs at individual interactions, which under different settings may be defined by different levels of shared proximity. This variety of settings results in variable infection rates and therefore different proportions of asymptomatic carriers.

An embodiment of the present disclosure provides a number of benefits, including:
(i) automates the task of communicable infection tracking and allows each individual in the population to accurately and immediately learn if they have been in contact with an infected person, or with an asymptomatic carrier;
(ii) can help to direct the needed self-isolation guidance to an individual following potential virus exposure and prior to developing symptoms;
(iii) can help to optimize the use of medical treatment and test resources for more effective infection detection and abatement;
(iv) can help to collect accurate and precise data for epidemiological research;
(v) can provide local, regional, state, and national authorities with dynamically updated information on disease distribution clusters and concentrations over the population.

An embodiment of the present disclosure takes advantage of data that is routinely collected related to movement of individuals according to information collected from their own personal communications devices that include an electronic control logic processor or central processing unit (CPU), such as, but not limited to, cellphones or smart phones, tablets or electronic pads, personal computers, smart watch wristband devices, or other portable or personal communications devices and can also include portable processors and systems resident in a motor vehicle, airplane, or other transportation vehicle. These processing and communications devices use wireless communication for electronic interaction with other devices and are typically provided with some type of location intelligence hardware for determining coordinates, such as the familiar GPS (Global Positioning System) or other type of GIS (Global Information System) tracking utility, WiFi, Bluetooth, or other wireless networking application. The collected information provides an accurate, time-stamped tracing of the precise geography of the corresponding communications devices and, by inference, of the location of the person who uses the device. Contact tracing data can also be obtained from other sources that collect data relating to an individual's geographical location or movements over a particular time interval, including records of ATM withdrawals, credit card purchases, ridership in public or private transportation, elevator use and timing (such as might be obtained from a security camera). In addition to a user's own vehicle, use of a bus or subway can also provide information and scheduling that include substantial data that can be useful for identifying interactions.

### The Tracing Problem

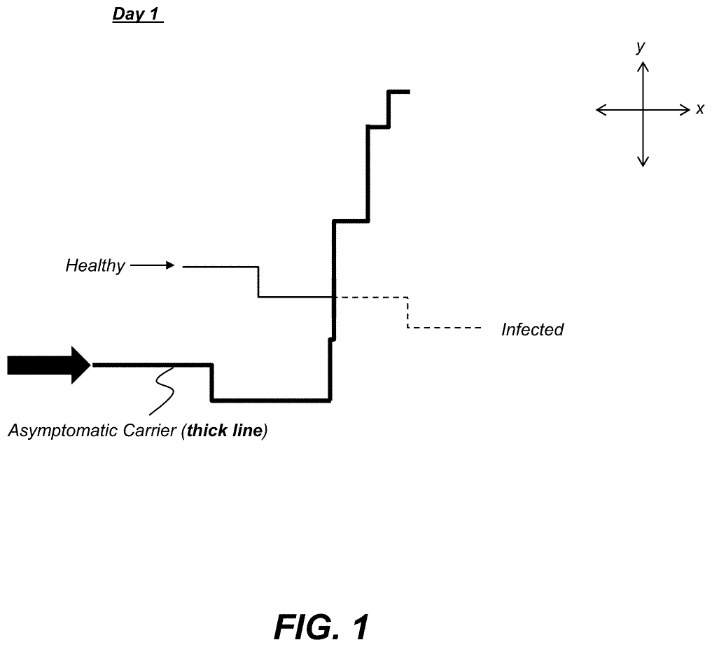

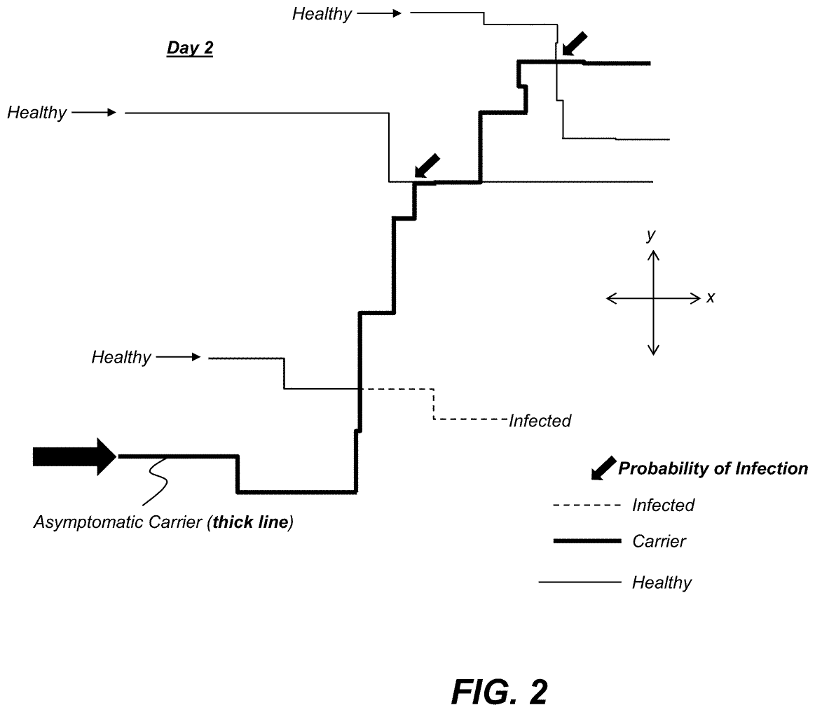

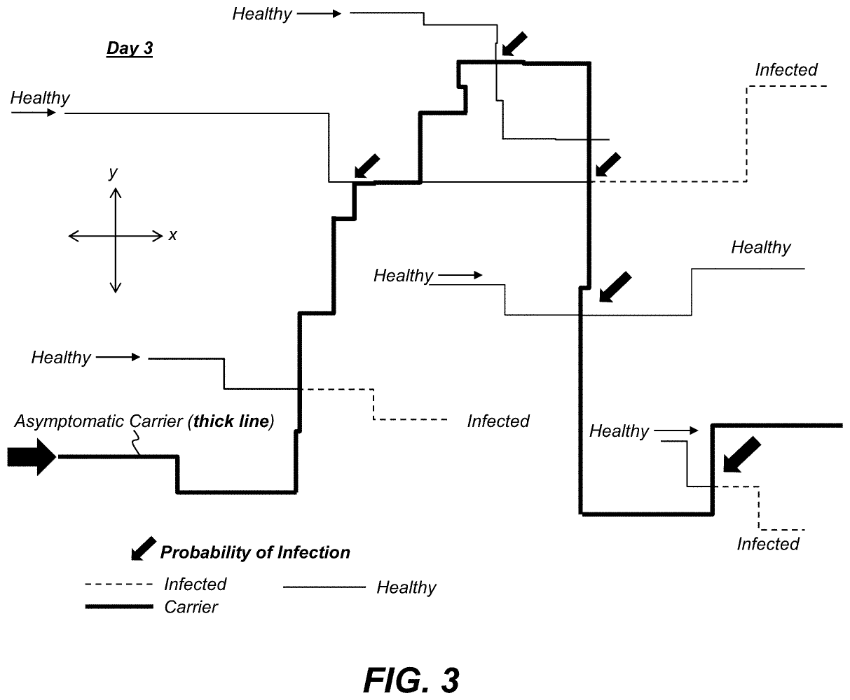

By way of illustrative example, FIGS. 1, 2, and 3 schematically show tracing of an asymptomatic carrier (thick line) and various contact instances of interaction with healthy or infected persons. The coordinate (x,y) axis shown represents geographical coordinates. FIG. 1 shows tracing of movement and geographic locations for the carrier on Day 1, with interaction with a healthy individual in visiting a first contact location. As can be seen, the healthy individual is later identified as infected. FIG. 2 shows carrier locations on Day 2, with tracked interactions with two other healthy individuals, neither of whom become infected. FIG. 3 shows the added information for Day 3, with interactions with one individual from Day 2 who is later found to be infected. Other healthy individuals may or may not be infected.

As FIGS. 1-3 suggest, there can be considerable complexity in tracing interactions between numerous individuals within a short time period. This type of complex problem can prove intractable to conventional algorithmic techniques, due to the huge and expanding number of variables and volume of location data points from which intelligence must be derived. To address this problem and provide accurate contact tracing and infection forecasting, the Applicants' solution applies a hybrid approach involving (as needed) statistical estimation and operations research modeling combined with machine learning to the conjoined body of location intelligence, generating data that would be used to reconstruct a contact “roadmap” similar to that shown in FIGS. 1-3.

An embodiment of the present disclosure can address the tracing problem by acquiring movement history recorded for individuals within a population, wherein the movement history for each individual includes a schedule of visited geographical locations and associated times at the visited geographical locations. The method identifies, from the acquired movement history, a set of one or more interactions between an individual identified as an infected carrier, symptomatic or asymptomatic, and one or more other individuals of the population. For each interaction in the set, a status of “exposed individual” can be assigned to each of the one or more other individuals in the interaction according to predetermined metrics of shared proximity with the infected carrier, which can be assumed or calculated, and overlapping time interval duration with the infected carrier at the geographical location. Overlapping time intervals would indicate, for example, two individuals in the same place for at least some portion of time; this could also indicate individuals within the same closed area within a period of time determined to be sufficient to allow infection to occur (such as successive riders in an elevator or vehicle, for example, wherein the overlap is determined according to how long an infectious agent may linger within a closed space after the carrier has moved on). Overlap can thus be determined according to relative temporal proximity as well as spatial proximity and may be adjustable using an administrative user interface, as described in detail subsequently. The method then transmits a signal with display content that alerts each exposed individual to an infection risk. The alert signal can also be transmitted to the carrier.

### Interaction Count and Weighting

An embodiment of the present disclosure uses interaction information from individual members of a population in a way which allows for several specificity levels for detecting asymptomatic individuals. For example, a rapidly spreading virus would result in a greater rate of asymptomatic transmission than other slowly growing pandemics. The Applicants method permits appropriate parameterization that gives accurate warnings to a community, regardless of the virus's rate of transmission.

The source of interaction data can be specified by system users or public health officials. As noted previously, for example, GPS locations or Bluetooth coordinate data can be used to quantify individuals' interactions with one another, as well as a simple questionnaire specifying place of work, residence or mode of transportation. Either option provides a quantifiable source of proximity measurements among individuals in a particular visit, and can be used to identify asymptomatic individuals and reduce the stress and strain on public healthcare systems by mitigating spread in a proactive manner.

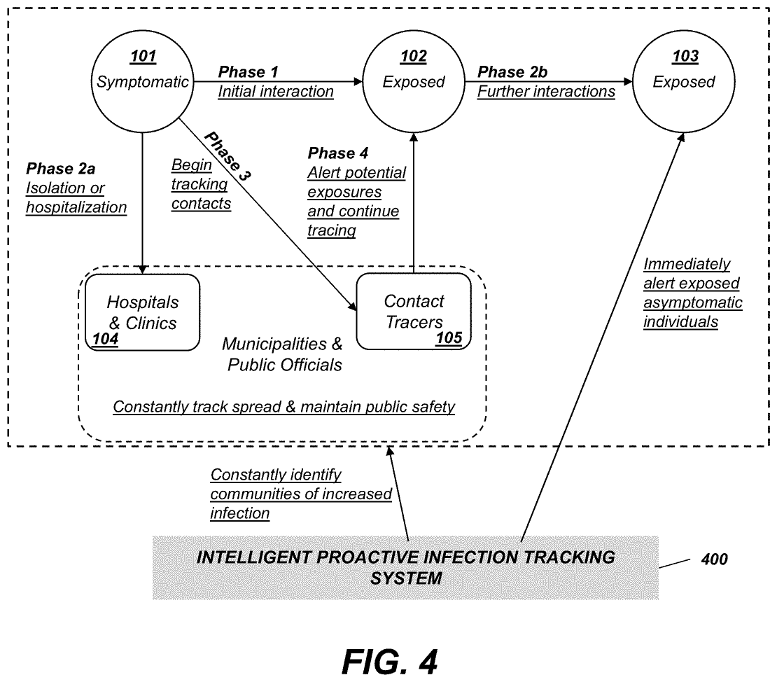

The logic flow diagram of FIG. 4 relates the response of an intelligent proactive infection tracking process 400 to conventional practice for interaction of key players in public health and municipalities within the general population to contact tracers and medical service providers. Individuals in a community, represented by the circular symbols, interact with each other, causing viral spread. For instance, an initial carrier individual 101 interacts with a fellow individual 102 and eventually becomes symptomatic. This process is followed by further interactions among the community as individual 102 interacts with another individual 103. Simultaneously, the original symptomatic individual 101 can be treated at a medical care site 104 or is isolated. As a response, contact tracing follows this admission as tracers 105 extract information from the initial carrier individual 101, such as information regarding others with whom individual 101 may have been in recent contact. These steps, representing the traditional and commonplace public health policies used to combat viral spread, can be seen to outline the weakness and blind-spots of current contact tracing methodologies. While contact tracers 105 in conventional practice identify and reach the potentially exposed candidate community member 102, the Applicant's proposed system 100 would have already gathered the necessary information to alert all potentially exposed members 102 and 103 of potential exposure and of being likely asymptomatic carriers.

The processes, methodologies and computational choices made to embody these aspects of the present disclosure may vary by setting and user. For example, some embodiments may only include communication channels between the system 100 and public health officials. Others may include direct notification methods from the system's analytics processes to individual community members. Some embodiments may include inputs such as medical tests or physical specimens when computing the likelihoods of individuals asymptomatically carrying a virus or other infectious agent or indicating which community members are infected. Other embodiments may only accept inputs from health providers and limit the input required from individuals in the population. As presented in this document, the Applicant's process allows for this flexibility in implementation while guaranteeing in its embodiment a system that identifies likely asymptomatic carriers within communities and the greater population, intelligently mitigating the spread of epidemics in a network of individuals.

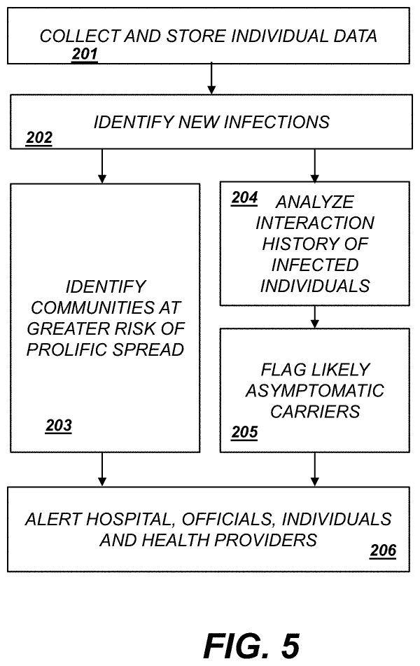

FIG. 5 is a flowchart displaying the steps and processes that improve the operational and medical alertness of the public health system. First, data is collected about members of the general population and specific communities in an information acquisition step 201. This collection can be tailored to specific groups, for example by gathering greater amounts of information about high-risk individuals such as the elderly the system can be less intrusive for the majority of the population. Alternatively, the same level of information could be collected from all individuals within a population. A set of interactions for processing and analysis can be obtained for this purpose.

The information obtained in step 201 can be stored in a variety of ways, both physically and digitally. For example, collection could be performed by an automatic program running on a single or a group of processors, with the acquired data immediately stored in a digital relational database. The database can be hosted on the same computerized environment or distributed among a network of devices.

Independently of the data acquisition process, but following it logically with the objective to find asymptomatic individuals among the population, symptomatic or confirmed infected cases are reported to the system embodying in an identification step 202. This reporting can be a part of the initial data collection process, or completely unrelated to it. According to an embodiment, individuals may be asked to manually report their own infection, while other embodiments may use third channels such as hospitals or medical laboratories as a source of information regarding new infections among a population; alternately, some combination of methods could be used.

Following the addition of this information, the data collected thus far allows the system to perform useful processes to assist in communicable infection control. One of such processes is the ability to identify communities at risk of greater infection rates in a locality identification step 203. These communities can be exemplified by office spaces, residential halls, commercial buildings and even modes of public transportation as well as other establishments. Any form of proximity which can be quantifiably defined by the system may establish a community of individuals. A second process that can proceed following data gathering data on infections is an interaction analysis step 204.

Following this, the system can analyze, then identify which specific individuals are likely to have asymptomatically spread the virus, identifying candidate asymptomatic carriers in an individual identification step 205.

The history of past interactions allows for this determination, as past infected individuals' recent interactions is available for the system to statistically rule on the likelihood of interactions to have caused exposure. These processes are lastly followed by the ultimate objective of the process and its main output, as it is capable of immediately alerting relevant actors, in a notification step 206 of flagged asymptomatic individuals and at-risk communities. The choice of relevant actors is of wide variety, as direct notifications can be sent to community members and individuals of these results, or a private alert can be forwarded to officials and municipalities without informing members of the general public. As soon as symptomatic cases or infections are confirmed in step 202, the system would be able to analyze which individuals have most likely caused infections without showing symptoms, and the communities which these individuals put at risk by their proximity can be alerted within seconds, as well as medical centers, public health officials and other individuals in preparation for a potentially upcoming greater load of infections.

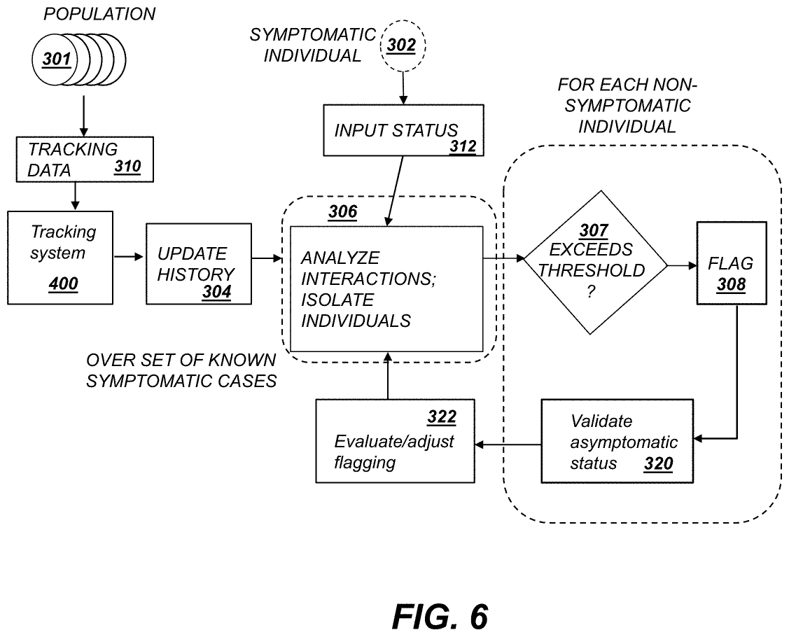

The flow diagram of FIG. 6 explains abstract processes presented in FIG. 5 and executed on a computer or other logic processor with greater detail, with particular focus on the back-tracking processes executed as part of interaction analysis step 204. While the figure only displays a snapshot of the information flow procedures, these processes can be executed on a continuous and regular basis in order to consistently alert individuals and health officials of increased risk. Members of the population, individuals 301, input tracking data 310 that is used to analyze proximity and possible infection into tracking system 400.

The granularity of this tracking information may vary from distinct physical location data collected as a set of transmitted signals in real time to less precise information such as a survey of recent travel or commuting destinations which individuals provide to the system, which may be some time after the fact. After this data is collected, interaction history is updated for the entire population in an update process 304.

### Defining Interaction

A definition of an interaction is required to make the determination of two individuals interacting, and such a definition may vary from setting to setting. For example, knowing the physical location of all individuals would allow the system to determine proximity and length of contact among members of the population, and a simple threshold of proximity can be used to define a potentially infecting interaction. Alternatively, an interaction may be defined as two individuals having shared a living space or mode of transportation for at least a predetermined time interval, and surveying the population's residential location or means of daily commuting can provide proximity criteria that determine these interactions.

Interaction identification is based on two variable factors that can be fine-tuned using machine learning to provide accurate results: (i) geographical location, typically defined from movement history in terms of spatial coordinates associated with a site; and (ii) time duration during which two or more individuals are at the same location. A factor of interest is the amount of overlap between the time interval for presence of a first individual at the location and the respective time interval for presence of the second person at that location. The time intervals can be adjusted, for example, to shorten or extend the time threshold beyond which exposure may be considered likely or highly possible, even including time after departure of a carrier, such as adding a few minutes to time interval duration at a particular location after the carrier has exited. As part of the geographical location, data as to proximity can also be significant, where it can be determined, for example, that a threshold distance was maintained between the two individuals at a particular site.

As an abstract example, assuming that there are two individuals in the population, the data gathered from them at any point in the process of information collection can be represented in a two-dimensional array. This could represent an environment wherein the only known information the system gathers for each individual is a location on a flat grid.

Using Location_{i,t} to be the site or location of individual i and time t, and defining an interaction as proximity of less than 5 units in Euclidean space between two individuals' locations, such as obtained from GPS coordinates, then the following location data can account for an interaction between individual 1 and individual 2:

Location_{1,t,}=(2,2), Location_{2,t}=(2,3)

A variable proximity threshold can be determined. In the example given, since the coordinate locations of the two individuals are less than 5 Euclidean units apart, then at time t, the system identifies an interaction between the two individuals. It can readily be appreciated that this distance measure is exemplary and can be increased or decreased according to need or based on determined accuracy. Shared proximity can also be determined where there are multiple carriers at a geographical location during the same time interval.

Such an example only serves to present one possible technique for interaction counting, and many others can be used to fulfill this requirement. For example, in order to avoid having to compute Euclidean distance among all members of a population, the space occupied by the data-vectors collected from each individual can be split into smaller parts, within which interactions can be detected. These smaller parts or “cells” can signify the boundaries of an office space, a public bus, an elevator, or other defined space, such as an apartment, house, dormitory, or other residential location. Such a division of the space would reduce the computation overhead required to identify interactions, since interaction related to infection would be concerned with individuals sharing a common cell for a predetermined duration. Resolution/granularity/cell boundaries

Regardless of the choice of proximity measurement and interaction definition, interaction of two individuals at time t can be stored after the interaction is positively identified. One method of such storage would account for both the specific details of the interaction such as time and place, which could be later used by the system to retroactively identify specific interactions both in time and space. An alternate embodiment would only account for the occurrence of the interaction and the individuals who caused it. Such an implementation would still allow for the eventual identification of possible asymptomatic carriers.

Interactions between candidate individuals can be recorded in a matrix M whose values M_{i,j} signify the quantity of interactions that individuals i and j have had throughout time. This quantity could increase by a constant amount at each interaction, or by a variable amount that changes with time. Different implementations of this method of accounting for interactions result in a variety of analysis methods as well as objective results in identifying higher communities at risk. For example, an interaction that occurred at time t₁ could have greater importance in the spread of the virus than an interaction at time t₀ if t₁>t₀ since it could be more likely for interaction to occur at a later time once the virus has spread more prolifically among the population. The opposite could be true as well, if the rate of infections is slowing within the population.

As an example, using the same Euclidean computation as previously to represent the space within which individuals move throughout time, consider two individuals, 1 and 2, interacting at time t=0, followed by individual 2 interacting with a third individual 3 at time t=1. If all interactions are given the same weight regardless of the time of their occurrence, the resulting matrix M would have the following form:

$${M}_{1} = \begin{matrix}\begin{matrix}[ [ 0 , 1 , 0 ] , \\ [ 1 , 0 , 1 ] ,\end{matrix} \\ [ 0 , 1 , 0 ] ]\end{matrix}$$

These examples present only one form of collecting, storing, and counting data that describes proximity and identifies interactions among a population; the Applicants' process is not limited to such characteristics. Many other quantitative forms of accounting for such information can be used to achieve the objective of determining which communities are at risk of greater infection rates and which members are likely to be asymptomatic carriers.

In simultaneous manner with respect to these processes and corresponding to identification step 202 in FIG. 4, symptomatic individuals 302 and other confirmed infected cases are reported to the system as input status data 312. The input channels providing this information to the system can vary from direct status update from individuals to third party laboratories and medical facilities which hold information regarding infectiousness and symptoms for various individuals. This data can be stored in a variety of ways. For example, the system may maintain both the identity of symptomatic and infectious individuals as well as the time at which they became infected or began to show symptoms. Other embodiments may only record the identity of the individual, and use other aspects of the system to allow for this information to identify potential asymptomatic individuals.

According to an embodiment of the present disclosure, a list or database of all known infected and symptomatic individuals is maintained. Consider, as an example, a population of 5 individuals characterized at time point t with the following interaction matrix similarly defined to the previous example:

$${M}_{t} = \begin{matrix}\begin{matrix}\begin{matrix}\begin{matrix}[ [ 0 , 1 , 0 , 0 , 4 ] , \\ [ 1 , 0 , 3 , 0 , 2 ] ,\end{matrix} \\ [ 0 , 3 , 0 , 1 , 1 ] ,\end{matrix} \\ [ 0 , 0 , 1 , 0 , 2 ] ,\end{matrix} \\ [ 4 , 2 , 1 , 2 , 0 ] ]\end{matrix}$$

If at time t, the known list of infected and symptomatic individuals is L=[1, 3], a quantitative measurement for the possibility of a candidate individual being asymptomatic can be computed in an analysis step 306. One version of this method sums the interaction count over all known individuals in the infected and symptomatic individuals database L. Such a method would produce a list or vector of interaction counters between each individual member of the population and all known infected cases. This vector can be referred to as v and this method of analysis displayed as follows:

$$v = {\sum }_{j ∈ L} {M}_{j}$$

wherein Mⱼ is the j^{th} row of the M matrix. For the example displayed above, this result vector would be:

### V=[0, 4, 0, 1, 5]

Other embodiments and implementations of the present disclosure may utilize different statistical methods to process interaction data for candidate individuals in combination with knowledge of infected population members. This allows for a variety of ways to arrive at such a result, which ranks members of the population based on their likelihood to be asymptomatic and pinpoints communities within the population that are at greater risk of rapid infection rates. In this example, the result of analyzing past interaction data with the addition of knowledge regarding infected and symptomatic cases gives the following ranking:

| Member of the | Score |
|---|---|
| population (i) | (vᵢ) |
| Individual 5 | 5 |
| Individual 2 | 4 |
| Individual 4 | 1 |

Such a result provides clear indication of which members of the population are most at risk, specifically of having asymptomatically infected others. Identifying these individuals is a key step toward mitigating future risk, reducing infection rates and easing the load on the healthcare system.

The system of the present disclosure would be able to act on this information and alert all relevant players and actors of such results. For example, the scores or infection risk values resulting from the example given above may be interpreted as a direct indicator of likelihood to be asymptomatic, and individuals with the highest scores or infection risk values would then present the greatest risk for causing future infections. The actions taken upon such a result may also vary from one implementation to another. In some situations, providing these results to the public may be of greater importance, allowing all individuals to know which individuals could pose the greatest risk of exposure. Calculation of a suitable risk value can be weighted by any of a number of factors that help to quantify the overall likelihood of infection and can be adjusted for factors of age, overall health, relative danger of the infection, and other factors deemed suitable by medical authorities.

In response to societal and municipal needs and norms, a different action may be more appropriate, and only revealing individuals their own scores in a private manner may result in more favorable outcomes. However, the method described herein allows for an even more robust and intelligent action, as healthcare systems may be automatically informed of the smartest action to take based on a classification algorithm. If the method for carrier identification results in a one-dimensional list resembling the example given above, a single threshold could be used to distinguish likely asymptomatic individuals from regular susceptible individuals. However, other implementations may use different methods of analyzing proximity and interaction data as well as identification of symptomatic individuals, and may give a different format of ranking the risk associated with non-symptomatic population members. Therefore, the classification may take several dimensions of inputs and deploy a variety of learning algorithms to identify risks such as communities with higher likelihood of greater infection rates.

Regardless of these choices, the methods of the present disclosure would permit those in authority, and those with visibility into the system, with ways for controlling the system's sensitivity to risk. Using the example above, a single threshold value X could be used in a decision step 307 to determine which values of v are correlated to likely asymptomatic carriers. For example, health officials utilizing this process could determine that 3 is the threshold score, past which individuals are most likely to have asymptomatically infected others. This would result in the flagging of individuals 5 and 2 from the table given previously as asymptomatic carriers in a flagging step 308.

Such a decision could cause a greater number of false positives (FP), and different settings may make it more desirable to reduce these while increasing false negatives. The sequence described herein allows for such a capability, giving precise control over its desired effect. Public health officials may adjust values used accordingly, with decisions to vary these values with time, such as in a validation step 320 and subsequent evaluation/adjustment step 322. For example, initial stages of spread may call for large FP numbers, assuring that no asymptomatic carriers are classified as non-carriers, while at later stages with lower infection rates, FP numbers can be lowered, allowing an increase in false negative values, as the importance of reducing the number of alerted and isolated members of the population grows. Using the example table provided earlier, this would mean that the threshold of 3 would be increased to 5, so that only individual 5 would be flagged as asymptomatic.

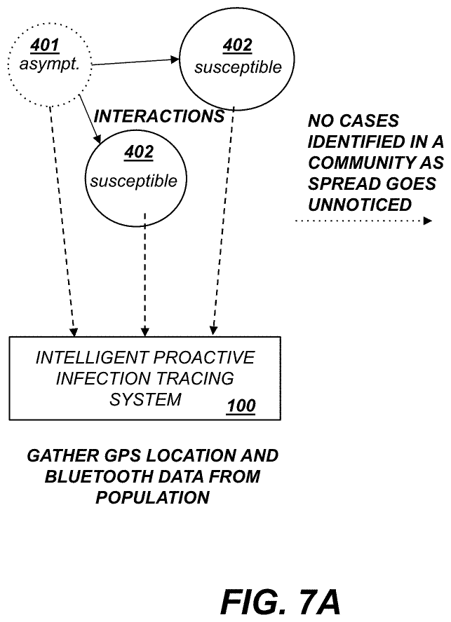

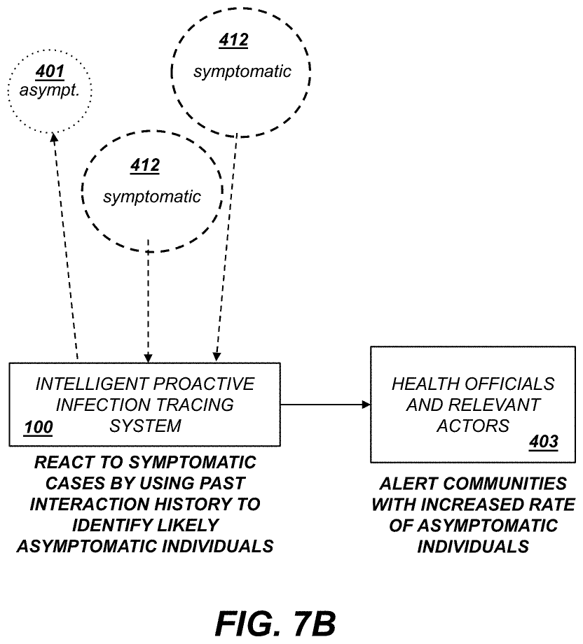

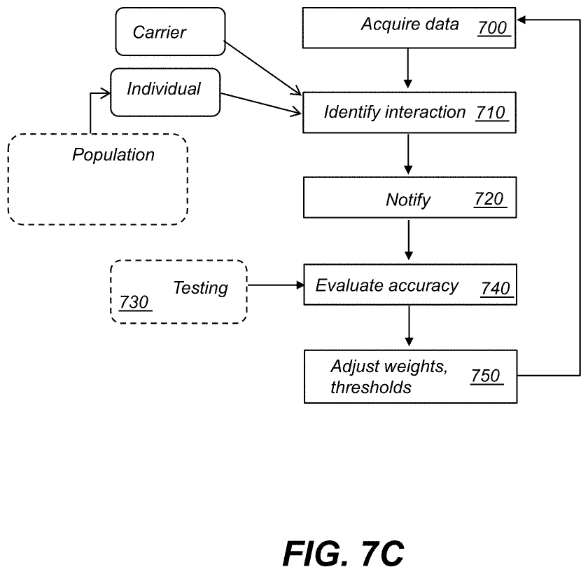

Processes and methods described herein represent possible embodiments of the present disclosure, showing some of the functions available at a particular time. FIGS. 7A and 7B are flowcharts describing the continuous utility of the Applicant's system as time progresses. As an example, a population of three individuals is used. One member is an asymptomatic carrier 401 and interacts with the other two at separate occasions, changing their status to susceptible individuals 402. As these exposures occur, system 100 continuously collects data on the individuals, as described previously. This data could be a set of signals representing GPS location, Bluetooth proximity or can use any other method of gathering movement and location information from the population.

As the outbreak continues, no signs of spread may show even though the population is very much at risk. This lack of information may cause individuals, health officials, and care providers to be unaware of a growing problem. At a later time, the two exposures described with reference to FIG. 7A may cause members of the population to show symptoms and become confirmed cases of infection, as shown in FIG. 7B. Even though the population did not expect this sudden development, the system 100 would have the interaction data necessary to identify the source of the risk, and can quickly act upon it to alert authorities 403 and individuals of who may be imposing a risk on their community, symptomatic individuals 412. The manner in which these developments occur and the manner in which the system's knowledge and capabilities are used may differ between implementations. This is only one potential example of such an implementation, and the data gathering as well as analytical aspects of its utility may require a different approach to information collection, processing, decision making and risk alerting in different cases.

FIG. 7C shows a logic flow diagram with steps for a method of contact tracing, showing the learning process performed by system 100 according to an embodiment of the present disclosure. The logic flow can be considered as a continuous process, executing in a type of loop as shown. A data acquisition step 700 obtains movement history data from wireless or cellular phone information or other records. An interaction identification step 710 identifies one or more interactions based on predetermined criteria for location proximity and duration. For a notification step 720, a classifier can be applied to the interaction data to notify a likely asymptomatic carrier based on interaction data. Testing provides results 730 that can be used to evaluate accuracy in an evaluation step 740. An adjustment step 750 then adjusts weight and threshold values used by the detection logic. The logic can use machine learning, conventional programming code, or a combination of coded instructions and artificial intelligence (AI) utilities for execution.

As system 100 continues to collect data, identify interactions and determine which individuals are asymptomatic, the system controlling and maintaining the execution of all these processes also learns to better classify asymptomatic individuals by routinely adjusting its specifications and classifications parameters in step 750. This can be done with the help of another data collection process which represents the verification of earlier classifications. An example procedure to perform this action can be based on the precision of the classifier. The precision is the ratio of the number of correctly flagged asymptomatic carriers to the number of total asymptomatic flags. In reality, not all the flagged individuals may get tested after the fact, but the precision of available asymptomatic carrier testing may provide sufficient statistical significance to improve future classifications. For example, if n is the number of correctly identified asymptomatic carriers and m is the number of incorrectly flagged carriers, then the precision p would be

$$p = \frac{n}{n + m}$$

A low value for p may indicate that too many individuals are incorrectly flagged as asymptomatic, which could be undesirable. A machine learning system embodying the invention, aware of its precision, could improve its execution by adjusting its threshold for classification, such that a greater score of past interactions with confirmed infected cases would result in flagging as asymptomatic. Inversely, if precision is excessive, this may indicate that the classifier is likely dismissing many asymptomatic cases as non-carriers; This may indicate that the threshold of classification is too high. Additionally, a precision value that is too high or too low could indicate that the proximity condition accounting for interactions and exposures could be too lax or too strict. If individuals continue to have many interactions with others that turn out to be symptomatic, yet they themselves continue to test as non-carriers, then too many interactions could be counted, and a modification in the proximity condition could be appropriate. Such changes to the system could be made automatically by the system itself as a constantly running procedure, or alternatively be left for supervisory users of the system to decide.

### Notification Strategy/Display

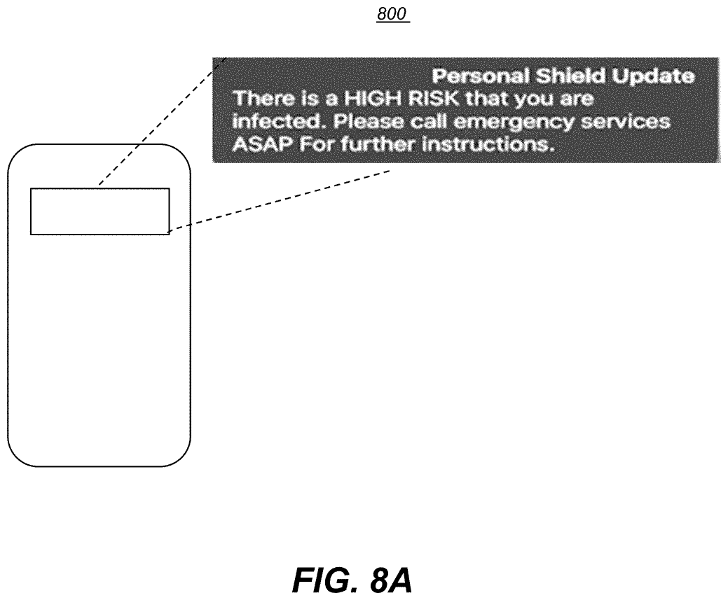

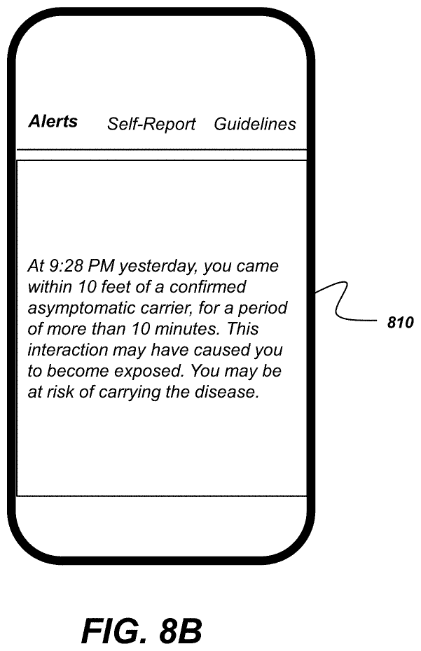

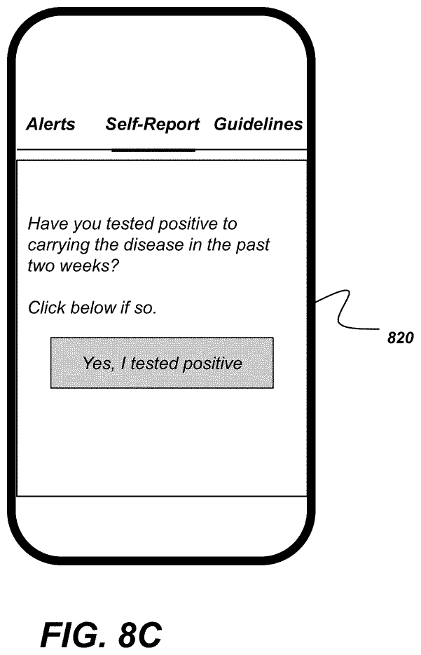

FIG. 8A is an example of a text message 800 that can be generated and transmitted as a potential notification display for an individual determined to be at risk, according to an embodiment of the present disclosure. After collecting data regarding individuals' interactions, and identifying infected members of the population, the system can determine, using its analytical and classification processes, if an individual is likely to be asymptomatic and may have infected others. Thus, a message indicating possible status as an asymptomatic carrier can alternately be displayed. Additionally, the system would have the information necessary to determine which communities within the population are at risk of upcoming outbreaks. This can be done by a variety of methods, for example by determining which subgroups within the population are showing several likely asymptomatic carriers. In either case, a notification or alert can be sent to each individual's mobile device, as shown for exemplary text messages in FIGS. 8A-8C. The alerts may contain more information than shown in the interface, and could be accessed through a different interface or device, such as using audible messages, such as phone messages or recorded voice messages, for example. FIG. 8B shows an explanatory message 810. FIG. 8C shows a prompt message 820 for testing verification by an individual.

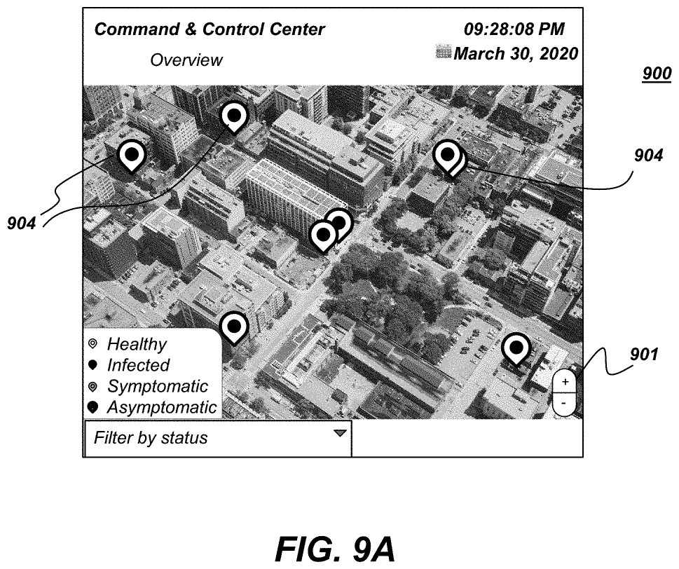

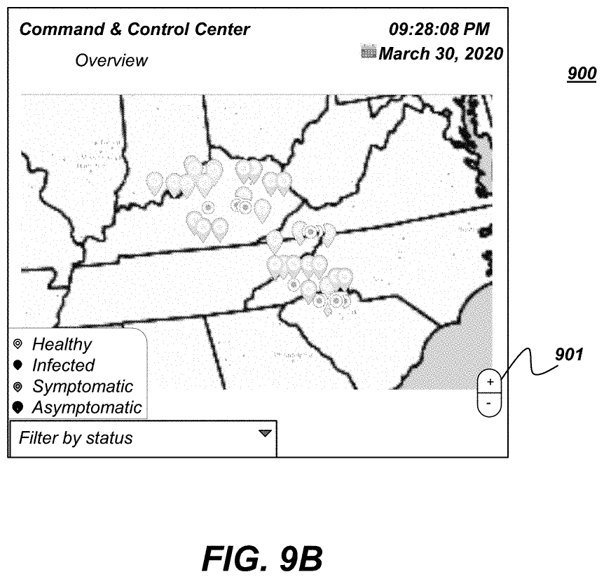

FIGS. 9A and 9B show an exemplary command and control configuration display 900 which displays the information gathered, processed, and analyzed by the system. This display can be generated to appear, for example, on a control monitor that is in signal communication with a processor executing system logic. In this “dashboard” display configuration, privileged users such as health officials would be able to view the geographical location of an infected individual, risk levels of different communities, and identities of potential asymptomatic members of the population, along with locations identified with icons 904. Such an interface could be updated in real time, alerting and notifying users of any relevant developments in the population. The scope of the command and control environment can vary by geography. For example, the image can show the status of a single neighborhood or can provide a control 901 that allows the user to zoom outward for a larger region as in FIG. 9B or to filter the information in any available direction, including viewing the risk status of multiple populations, cities, counties, states, or nations, for example.

Temporal filtering can also be performed, allowing the viewer to revert to an earlier set of conditions, such as previous day, month, or year, for example. Using this capability, the viewer can observe the risk levels of different communities at previous stages in an outbreak. Various embodiments allow for such capabilities and features. Another filtering choice available to users of the system would be to highlight individuals in different groups, separately or simultaneously. The risk analysis procedures would identify potential asymptomatic individuals, while the data collection processes would continuously track susceptible members of the population. Users controlling the system would be able to both view the most recently known data about these members as well as the most recent risk level analysis results, all at the same time and through the same visual frame, display 900.

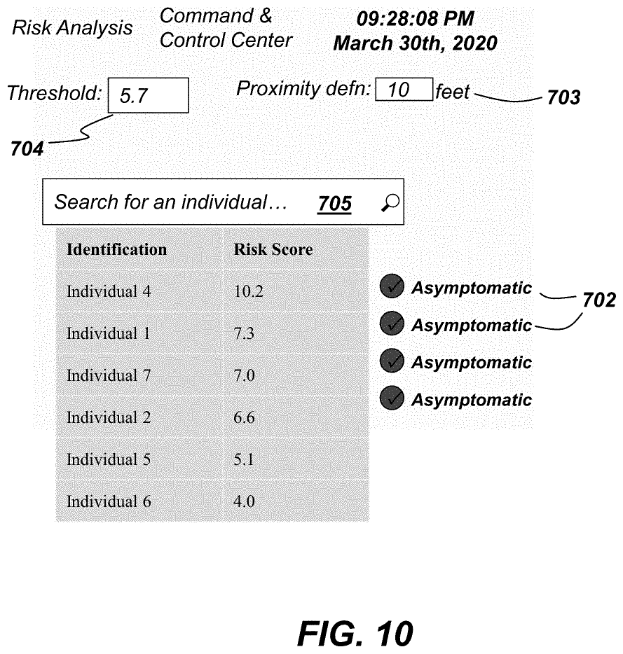

FIG. 10 is an interface display that shows a summary of risk analysis results for the population. The example interface shows a table in which users of the system, particularly administrative users, can view risk levels associated with each individual. As described in an earlier example, this risk can be quantified by a one-dimensional quantity such as a number or score which indicates the likelihood an individual is an asymptomatic carrier, based on the individual's past data and interaction history. For those individuals who are likely to be asymptomatic, the interface could display, within the same frame, identities of members flagged as asymptomatic carriers, using a label 702. In the same interface, a user would be able to control system parameters such as the proximity criteria 703.

An embodiment of the present disclosure provides default criteria for determining what constitutes an interaction, but allows user editing to specify criteria to be used at a particular site. The criteria and values can be user selected, with no inherent definition provided. The selection can depend on what information is available about individuals in the population.

In the example depicted in FIG. 10, it is assumed that the distance between individuals is known from the accessible data, and so defining a proximity condition based on distance would change the number of interactions community members have with each other. Such a choice would, as a result, give different risk level analysis as well as asymptomatic classifications. Additionally, the threshold infection risk value 704 for classifying an individual as asymptomatic could be varied through such an interface. This would give the user the choice of specificity when it comes to the rate at which individuals are flagged.

As mentioned earlier, different settings and conditions may require more or fewer individuals to be flagged as asymptomatic. Another capability such an interface can offer is a search utility 705, providing the ability to locate specific information on an individual member of the population and display this information, as well as risk level. This is only one possible way to display and interact with the information stored and processed by the system. It can be appreciated that there are numerous possibilities for accessing and viewing the many procedures, processes, logic, and database tables that are available using system 100.

According to an embodiment of the present disclosure, contact tracing of a carrier of a communicable infection within a population can particularly help in identifying the asymptomatic carrier of infection. A symptomatic carrier can initially be identified according to received medical data. Recorded tracking information that shows geographical location and timing for each of a plurality of members of the population within a predetermined time period, such as wireless phone data, for example, can be acquired. From this acquired information, a location visited by the carrier, along with an associated time interval for this visit, can be obtained. Then, overlap for any other time period for a second individual at the same site can be determined and a corresponding infection risk value for the second individual calculated accordingly. The system can respond by transmitting a signal with message content that alerts the second individual to the calculated infection risk value. The message content can display on a personal communications device associated with the second individual, including with a suitable public health dashboard.

Intelligent proactive infection tracking system 100 can be executed by a processor that is configured by programmed instructions to execute learned logic to perform the following functions:

a) identify a carrier of a communicable infection according to received medical data;

b) access recorded tracking information that shows geographical location and timing for each of the members of the population within a predetermined time period;

c) identify, from the recorded tracking information, at least a first geographic location visited by the carrier and a corresponding first time interval for the carrier visit;

d) identify, from the acquired tracking information, at least a second individual determined to have been present at the first visited geographical location for a second time interval that at least partially overlaps the first time interval;

e) calculate a corresponding infection risk value for the second individual according to an overlap of the first and second time intervals;

f) transmit a first signal having message content that alerts the second individual to the calculated infection risk value;

g) display the transmitted message content on a personal communications device associated with the second individual; and

h) display, on a control monitor that is in signal communication with the processor, a summary listing showing at least the second individual and the corresponding calculated infection risk value. This can include a message indicating asymptomatic carrier status to the second individual.

### Use of Machine Learning

To provide the needed computation for identifying asymptomatic individuals, the Applicants' approach is to execute statistical estimations and operations research modelling combined with unsupervised artificial intelligence (AI) logic to detect asymptomatic carriers in the general population. A key analytic idea is to track the dynamic history of entities representing all ‘positively’ infected individuals that are known at any point in time. At any chosen stoppage point T, the hitherto known history of all isolated individuals gives rise to a vector representing the likelihood of other individuals being asymptomatic carriers. New data constantly updates the various Bayesian classification parameters in this system. The method is functionally designed and controlled to minimize the probability of ‘False Positive’ (FP) alerts, while keeping the probability of ‘False Negative’ (FN) indications below α, a threshold setup by the systems manager. In this way, the machine logic can be continually learning and providing updated information relative to contact tracing for individuals.

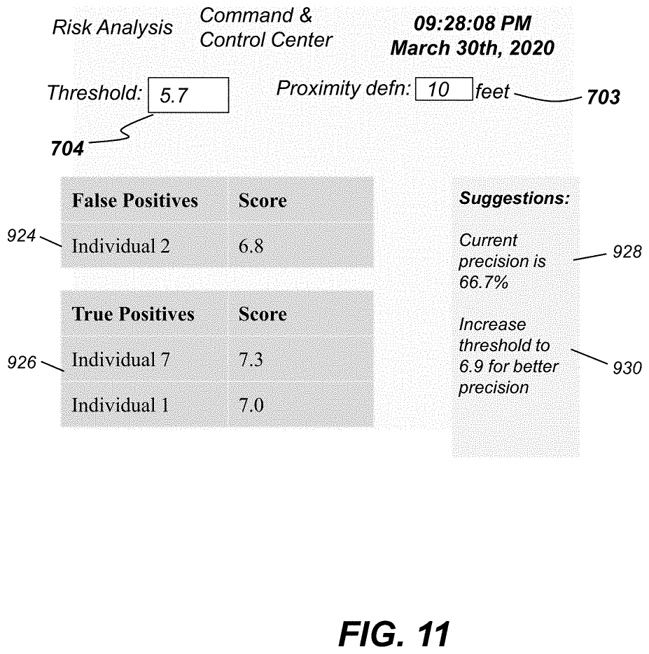

FIG. 11 is a control screen that can be displayed for allowing a system administrator or other user to make manual adjustment to threshold or other values based on results statistics. A table 924 can provide statistical values as well as individual designations for false positive results obtained by the system. Another table 926 can provide statistical values as well as individual designations for true positive results obtained by the system. Displayed suggestion text messages 928 and 930 can provide information on overall system precision and recommendations for adjustment generated by system logic. The processor executing system logic and providing the control screen of FIG. 11 on a control monitor can accept operator instruction entry to change variables such as the threshold value 704 or proximity criteria value 703.

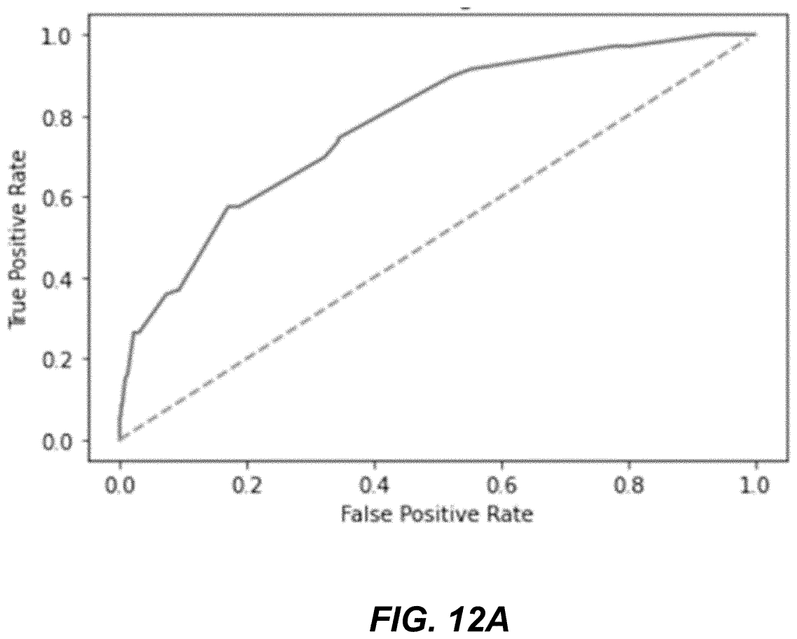

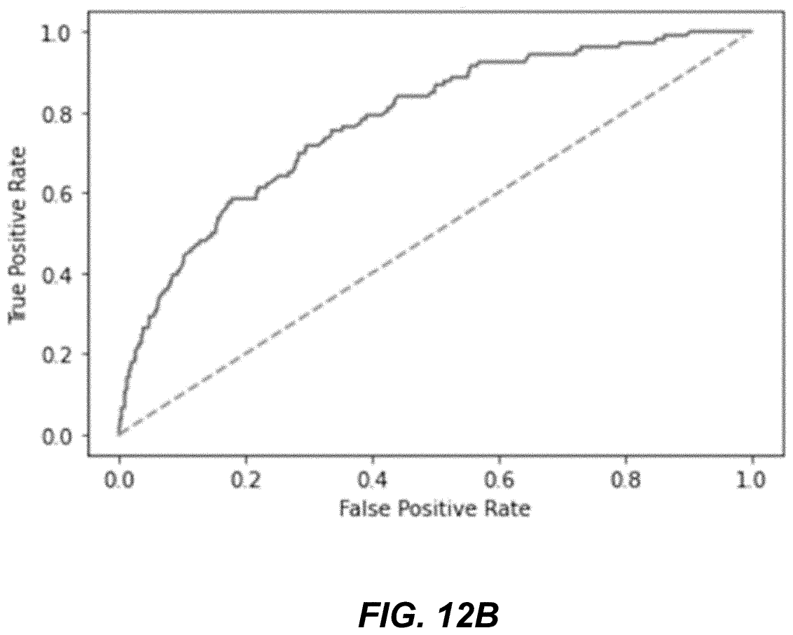

FIGS. 12A and 12B show ROC (Receiver Operating Characteristic) curves of selected classifiers from simulated testing using the Applicants' approach. The ROC curve plots the true positive rate (TPR) against the false positive rate (FPR). In the context of the present disclosure, the true positive (TP) rate is the proportion of correct identifications for an asymptomatic carrier of all positive identifications (TP/(TP+FN)), wherein FN is false negative identifications. Similarly, the false positive rate is the proportion of incorrect identifications as positive out of all negative identifications (FP/(TN+FP)) wherein TN indicates true negatives.

The ROC curve shows the trade-off between sensitivity (or TPR) and specificity (1−FPR). Classifiers that give curves that extend closer to the top-left corner indicate a better performance. As a baseline, a random classifier is expected to give points lying along the dashed line diagonal (FPR=TPR). The closer the curve comes to the 45-degree diagonal of the ROC space, the less accurate the test. The ROC can be particularly useful for evaluating classifiers used in predicting events such as disease infection.

The machine learning logic itself can execute on any computer processor that is capable of the needed data access, computational, and storage resources required for the application, including Cloud processing, for example.

The personal communications device associated with each individual serves as a vehicle for reporting position and time-stamp data. This automatic reporting is typically independent of any downloaded application, such as a cell phone app, provided for communicable infection notification and control. Thus, participation in application benefits is voluntary; however, the information that is collected and available for individuals can be obtained from data that is inherently available relating to users of cellphones and other portable devices that use some form of wireless communication. No downloaded application is needed; however, there can be a downloaded app that is provided as part of the system 100, with appropriate functions for individuals within the population, whether the user is simply a tracked participant or serves in a position of health care authority or provider.

Using the application provided in the Applicants' solution, the processor device is configured according to programmed instructions to serve as an input device for login and use of application resources and as an output device for display of risk notification and other useful information related to infection control and, optionally, overall health of the participating user.

An aspect of contact tracing that is not provided in conventional practice but can be provided using the Applicants' solution relates to methods for working back through the tracking data to identify asymptomatic carriers with high probability of success, based on detected instances of infection and continuously updated contact interaction data. Thus, for example, by detecting patterns of infection, the machine learning logic that executes on the Applicants' processor can systematically identify candidate individuals who may be unwittingly acting as carriers of the infection. Statistical thresholds can be used to evaluate the likelihood that a particular candidate is an asymptomatic carrier. As data continues to be collected, the infection pattern may reveal an increased probability that a person is an asymptomatic carrier.

The example tracing of FIG. 3 illustrates how an asymptomatic carrier can be detected using historical data. Statistics collected on interactions of a candidate with persons known to be infected at some later time can be used to generate probability data of carrier status for that candidate. By working back through contact instances (the slanted arrows in FIGS. 1-3), statistical data for calculating probability can be generated and re-computed in ongoing fashion.

By controlling one or more threshold settings for statistical probability, individuals can each monitor the likelihood that they themselves are serving in this role. Similarly, a public health authority, or other duly authorized governmental authority such as a designated municipal government official, may be able to take steps to intervene at some threshold and notify a particular individual that testing or isolation may be prudent or necessary.

### Risk Notification and Recording

An aspect of the present disclosure relates to risk notification options that can be provided using the Applicants' system. According to an embodiment, an app running on the personal communications device can run continuously or execute as requested by the user to invoke tracing logic and notify the user of risk assessment and action where necessary.

An application can continuously estimate the risk of each user's exposure based on their movement or location history, their personal medical risk factors, and the infection status and movement or location history reported for other users. As shown in the example of FIG. 5, a notification can be transmitted to the user's personal communications device and can provide guidance on recommended procedure based on the algorithm findings. For example, the user can be asked to immediately self-isolate and/or to seek medical guidance.

In addition to user notification, the user application can also provide data to a database maintained by local, regional, state, or national authorities, such as to add one or more data points to a map display, as shown in the example display of FIGS. 9A, 9B.

Users who obtain certified positive test results can be encouraged to update status through the app. This can help to facilitate identification of others with whom a user has been in contact, while helping to preserve anonymity, for example. The software can update other users who may have had contact with, or have been in the proximity of, the infected individual. This can help to provide insight into the infection network by identifying other users exposed to the virus through an asymptotic intermediary who may have been in contact with an affected individual.

Anonymity can be protected using an application envisioned by the Applicants, reserving personal information and sharing only anonymous data points with a disease control center or other authority. FIG. 7 shows an exemplary report that can be provided showing test results related to individuals.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

It will be apparent to those skilled in the art that various modifications and variations can be made without departing from the spirit or scope of the invention. Since modifications combinations, sub-combinations and variations of the disclosed embodiments incorporating the spirit and substance of the invention may occur to persons skilled in the art, the invention should be construed to include everything within the scope of the appended claims and their equivalents.

1. A method for contact tracing of a carrier of a communicable infection within a population, the method comprising:
a) identifying the carrier of the infection according to received medical data;
b) acquiring recorded tracking information that shows geographical location and timing for each of a plurality of members of the population within a predetermined time period;
c) identifying, from the acquired tracking information at least a first geographic location visited by the carrier and a corresponding first time interval for the carrier visit;
d) identifying, from the acquired tracking information, at least a second individual determined to have been present at the first visited geographical location for a second time interval that at least partially overlaps the first time interval;
e) calculating a corresponding infection risk value for the second individual;
f) transmitting a first signal having message content that alerts the second individual to the calculated infection risk value; and
g) displaying the transmitted message content on a personal communications device associated with the second individual.

2. The method of claim 1 further comprising identifying the second individual as an asymptomatic carrier and transmitting a second signal notifying the second individual of asymptomatic status.

3. The method of claim 1 further comprising transmitting a third signal with information related to the identified infection risk value for use by a governmental authority.

4. The method of claim 1 wherein the recorded tracking information is acquired from the personal communications device associated with the carrier.

5. The method of claim 1 wherein displaying the transmitted message content comprises displaying a text message.

6. The method of claim 1 further comprising transmitting an audible message to the second individual.

7. The method of claim 1 wherein calculating the corresponding infection risk value comprises computing proximity of the second individual to the carrier at the first visited geographical location.

8. The method of claim 1 further comprising generating a user display on a control monitor, wherein display content comprises:
(i) for at least the second individual, identifying text and the calculated corresponding infection risk value; and
(ii) a first variable value that is used in calculating the corresponding infection risk value.

9. The method of claim 8 wherein the first variable value relates to proximity to the carrier at the geographical location and is editable by the user.

10. A processor that is configured by programmed instructions to:
a) identify a carrier of a communicable infection according to received medical data;
b) access recorded tracking information that shows geographical location and timing for each of a plurality of members of the population within a predetermined time period;
c) identify, from the recorded tracking information, at least a first geographic location visited by the carrier and a corresponding first time interval for the carrier visit;
d) identify, from the acquired tracking information, at least a second individual determined to have been present at the first visited geographical location for a second time interval that at least partially overlaps the first time interval;
e) calculate a corresponding infection risk value for the second individual according to an overlap of the first and second time intervals;
f) transmit a first signal having message content that alerts the second individual to the calculated infection risk value;
g) display the transmitted message content on a personal communications device associated with the second individual; and
h) display, on a control monitor that is in signal communication with the processor, a summary listing showing at least the second individual and the corresponding calculated infection risk value.

11. The processor of claim 10 further configured to accept, on the control monitor, an operator instruction entry of a variable that changes the calculated infection risk value.

12. The processor of claim 10 wherein the programmed instructions execute machine learning.

13. The processor of claim 10 wherein the recorded tracking information comprises wireless phone tracking data.

14. A method comprising:
(a) acquiring movement history recorded for a plurality of individuals within a population, wherein the movement history for each individual includes visited geographical locations and associated times at the visited geographical locations;
(b) identifying, from the acquired movement history, a set of one or more interactions between an individual identified as an infected carrier and one or more other individuals of the population;
(c) for each interaction in the set, assigning a status of exposed individual to each of the one or more other individuals in the interaction according to predetermined metrics of shared proximity to the infected carrier and duration with the infected carrier at the geographical location; and
(d) transmitting a signal having display content that alerts each exposed individual to an infection risk.

15. The method of claim 14 further comprising, for one or more interactions:
calculating an infection risk value associated with each of the one or more other individuals in the interaction; and
displaying, on a display monitor, an identification of each of the one or more other individuals in the interaction, along with the corresponding infection risk value.

16. The method of claim 14 wherein the acquired movement history comprises data obtained from wireless phone tracking.

17. The method of claim 14 wherein shared proximity is calculated according to Global Positioning System coordinates.

18. The method of claim 14 wherein the display content comprises a text message.

19. The method of claim 14 further comprising transmitting an audible message that alerts each exposed individual to an infection risk.

20. The method of claim 14 wherein the infected carrier is identified as asymptomatic from the acquired movement history by detecting a pattern of infection through the population that shows, for one or more other infected individuals, a common interaction with the infected carrier.
